(19) United States
(12) Patent Application Publication
(10) Pub. No.: US 2026/0248754 A1
(43) Pub. Date: Aug. 27, 2026

(54) BUTYRATE SUPPLEMENTATION TO MAMMALS TO IMPROVE COGNITIVE AND PSYCHOLOGICAL FUNCTION

(71) Applicant: NutriScience Innovations LLC, Milford, CT (US)

(72) Inventors: Edward Dosz, Winfield, IL (US); Michael Lelah, Arlington Heights, IL (US)

(21) Appl. No.: 19/545,416

(22) Filed: Feb. 20, 2026

Related U.S. Application Data
(60) Provisional application No. 63/762,713, filed on Feb. 25, 2025.

(51) Int. Cl. A61K 31/22; A61K 9/00; A61K 47/36; A61P 25/22

(52) U.S. Cl. CPC A61K 31/22; A61K 9/0056; A61K 47/36; A61P 25/22

(57) ABSTRACT

The present disclosure relates generally to the supplementation of butyrate in the diet of mammals. Specifically, the present disclosure relates to a method for supplementing butyrate in the diet of a mammal resulting in improved physiological outcomes through the gut-brain axis, including improvements in anxiety and depression, and other cognitive behaviors, such as brain function, cognitive performance, executive function, stress, sleep, and focus. The present disclosure and methodology demonstrates advantages of administering a direct butyrate generating supplement, to influence the gut-brain axis through two pathways: a first pathway of butyrate generation leading to improvements in gut health which lead to improvements in brain health, and a second pathway of direct butyrate generation in the gut directly leading to improvements in brain health, independent of gut health.

### TECHNICAL FIELD

The present disclosure relates generally to the supplementation of butyrate in the diet of mammals. Specifically, the present disclosure relates to a method for supplementing butyrate in the diet resulting in improved physiological outcomes through the gut-brain axis, including improvements in anxiety and depression, and other cognitive behaviors, such as brain function, cognitive performance, executive function, stress, sleep, and focus. The present disclosure and methodology demonstrates advantages of administering a direct butyrate generating supplement, to influence the gut-brain axis through two pathways: a first pathway of butyrate generation leading to improvements in gut health which lead to improvements in brain health, and a second pathway of direct butyrate generation in the gut directly leading to improvements in brain health, independent of gut health, with a fast-acting response.

### BACKGROUND

Butyrate, a short-chain fatty acid (SCFA), is well known for its systemic benefits, including its role in metabolic regulation, immune function, and inflammation control. However, its effects on the gut-brain axis are less well studied, with emerging research beginning to uncover its influence on brain health and neurological function. The gut-brain axis facilitates the interaction between the enteric nervous system (ENS), which exists within the gastrointestinal (GI) tract, and the central nervous system (CNS). The ENS is involved in the regulation of basic gut functions and has similar neurotransmitters and signaling molecules as the brain. A key neural pathway for this bidirectional communication is the vagus nerve, which has a significant role in modulating the hypothalamic-pituitary-adrenal (HPA) axis, a regulator of the body's adaptive stress response.

Other pathways through which the gut and the brain communicate involve direct or indirect signaling via biochemical signaling. These chemical transmitters may include microbial metabolites, hormones, or neurotransmitters which can be directly synthesized or modulated by gut microbiota or introduced through the diet and supplementation. The short chain fatty acids, including butyrate, are very important in this regard, as they contribute to intestinal barrier integrity, mucus production, and inflammation regulation, key factors in maintaining gut and brain health.

Butyrate is a key SCFA produced endogenously by gut bacteria during the fermentation of dietary fiber and can also be supplemented exogenously. Butyrate interacts with various receptors attached to most cells throughout the body. Although the mechanisms of how butyrate regulates biochemical activity in these cells is just beginning to be investigated and understood, the fact that most cells in our body have butyrate receptors demonstrates its importance in health.

There is mounting evidence of a relationship between butyrate and the brain/nervous system. While the exact mechanisms have not been fully elucidated, free fatty acid receptors (FFARs), the SCFA-sensitive receptors, have been found on many nerve and brain cells, suggesting that butyrate may directly interact with the brain and/or nervous system as a signaling molecule. Beyond direct signaling, several indirect mechanisms have also been described. One of these is butyrate's activity as a histone deacetylase (HDAC) inhibitor. Studies have shown that when butyrate inhibits the HDAC enzymes in brain cells, genes that code for oxidative stress resistance and neurotrophic factors are upregulated. Butyrate has also been shown to positively enhance development and homeostasis of microglia, the immune cells specific to the brain and nervous system. Microglia help regulate neuroinflammation and maintain cognitive function. Higher levels of butyrate have been associated with increased neurogenesis, while research suggests potential benefits in neurodegenerative diseases such as Alzheimer's, Parkinson's, and multiple sclerosis. Through its neuroprotective effects, reduction of neuroinflammation, and enhancement of neuroplasticity, butyrate continues to show promise for brain health.

Butyrate is not intrinsic to humans or other mammals; instead, it is generated by the gut microbiota through fermentation of fibers or supplemented in the diet. ButyraGen® (NutriScience Innovations, LLC) is a patent pending, novel dietary ingredient that directly generates butyrate. Its primary active component is tributyrin, a short chain triglyceride, with additional active components including Sunfiber®, a partially hydrolyzed guar fiber, and other fermentable fibers (described in U.S. Patent Application Publication No. 2022/0269427, which is incorporated herein by reference in its entirety).

Therefore, a need exists for enhancing physiological improvements in gut health through supplementation of butyrate in the diet of a mammal leading to a corresponding reduction in anxiety and psychological discomfort, as well as improvements in other brain functions such as cognitive performance, executive function, stress, sleep, focus, etc.

The present disclosure provides supplementation of butyrate through a butyrate generator in the diet of a mammal to provide physiological improvements in gut health, including reduced digestive distress, which leads to a corresponding reduction in anxiety and psychological discomfort.

The present disclosure further provides supplementation of butyrate through a butyrate generator in the diet of a mammal to provide physiological improvements independent of gut health.

The present disclosure further provides a method for supplementing butyrate in the diet resulting in improved physiological outcomes through the gut-brain axis, including improvements in anxiety and depression, and other cognitive behaviors, such as brain function, cognitive performance, executive function, stress, sleep, and focus.

### SUMMARY

The present disclosure relates generally to the supplementation of butyrate in the diet of mammals. Specifically, the present disclosure relates to a method for supplementing butyrate in the diet resulting in improved physiological outcomes through the gut-brain axis, including improvements in anxiety and depression, and other cognitive behaviors, such as brain function, cognitive performance, executive function, stress, sleep, and focus. The present disclosure and methodology demonstrates advantages of administering a direct butyrate generating supplement, to influence the gut-brain axis through two separate pathways: a first pathway of butyrate generation leading to improvements in gut health which subsequently lead to improvements in brain health, and a second pathway of direct butyrate generation in the gut directly leading to improvements in brain health, independent of gut health, with a fast-acting response.

To this end, in an embodiment of the present disclosure, for supplementing butyrate in a diet of a mammal to promote improved physiological outcomes, is provided. The method comprises the steps of providing a dietary supplement composition formulated to increase generation of butyrate in a gut of the mammal, administering the dietary supplement composition to the mammal, wherein the dietary supplement produces a therapeutically effective increase in the amount of butyrate in the gut of the mammal for improved physiological outcomes.

It is, therefore, an advantage and objective of the present disclosure to provide improved compositions and methods of making and using the same for improved physiological outcomes.

Specifically, it is an advantage and objective of the present disclosure to provide improved compositions and methods for supplementing butyrate in the diet of a mammal to positively influence gut/brain health.

Moreover, it is an advantage and objective of the present disclosure to provide improved compositions and methods for supplementing butyrate in the diet of a mammal to positively influence brain health independent of any gut disorders.

### BRIEF DESCRIPTION OF THE DRAWINGS

FIG. 1 illustrates temporal (week-to-week) changes in Anxiety 4A male participant improvement over the course of the 6-week study;

FIG. 2 illustrates temporal (week-to-week) changes in Depression 4A participants with No GI Disorder improvement over the course of the 6-week study.

### DETAILED DESCRIPTION

Fiber provided by the diet may act indirectly through fermentation by gut microbiota which generates butyrate and short chain fatty acids and can potentially operate independent of exogenous butyrate generation in the same manner. Fiber ferments in the lower GI to produce butyrate. Butyrate formed in the lower intestine is either used by colonocytes for energy or discharged in the stool. On the other hand, butyrate introduced by supplementation is absorbed in the upper gastrointestinal track, where it can be absorbed directly into the portal vein and transported to the brain and systemically through the body, providing a different mechanism for supplemented butyrate.

The present disclosure found a statistically significant improvement in anxiety and depression over placebo with butyrate supplementation. This means that butyrate supplementation produces clinically significant effects over and above that produced from diet alone.

The present disclosure includes various forms of butyrate supplementation to the diet including sodium butyrate and butyrate salts, butyrate attached to other molecules, such as lysine-butyrate, ex-vivo activated probiotics, e.g. activated with fiber to ferment and generate butyrate and other short chain fatty acids which are then fed to humans and animals and including where the probiotic bacteria are alive or dead, and direct butyrate generators, such as tributyrin which generate butyrate in the human or animal body.

To demonstrate the proposed advantages of butyrate supplementation, a human clinical randomized, placebo-controlled, cross-over pilot study in healthy adults with no digestive issues was previously conducted to assess the tolerability and determine mechanisms of action of ButyraGen®. This 1-week placebo, 3-week active cross over study, demonstrated that ButyraGen® was safe and well tolerated. Butyrate in the blood was undetectable, possibly due to its transient nature or the low dose used in the supplement. However, secondary effects of butyrate generation and absorption were observed, including significant increases in triglycerides and non-significant decreases in hs-CRP and blood glucose. These findings were indicative of downstream effects of butyrate generation, metabolism, and adsorption into the blood. A proposed mechanism of action involves butyrate generation in the small intestine by the action of pancreatic enzymes on tributyrin, followed by absorption into the portal vein and then activity in the liver.

The impetus behind the present disclosure was to examine the effects of ButyraGen® consumption on the gut-brain axis in a real-world setting using a supplement-level dose. An objective of the present disclosure was to further investigate the role of ButyraGen® supplementation in supporting various aspects of anxiety and non-clinical depression through a 6-week, 2-arm, randomized, double-blind, placebo-controlled clinical trial. In the present disclosure a series of validated questionnaires were used to assess stress and brain health and their impact on overall quality of life. This disclosure aims to address critical gaps in understanding the benefits of butyrate supplementation at practical doses on the gut-brain axis, potentially offering a new avenue for improving brain and gut health in healthy and at-risk populations.

### Experimental Methodology

Clinical Study Overview: This randomized, double-blind, placebo-controlled clinical study was conducted by Radicle Science Inc, Del Mar, CA, USA (study number RADX-P-2407) as part of a combined GI and psychological assessment study. The GI specific results have been reported elsewhere. This study adhered to the International Conference on Harmonisation guidelines for Good Clinical Practice (ICH E6) and the Code of Federal Regulations of the Protection of Human Subjects (45 CFR, Part 46), including informed consent, which was obtained from all participants. The study evaluated the safety and effects of ButyraGen® on self-reported anxiety and non-clinical depression health outcomes starting with approximately 600 adults aged 21 years and older that reside in the USA. The study was designed as a direct-to-consumer, virtual trial with no in-person visits. Participants were randomly assigned to either the active or placebo group and consumed their assigned product daily for 6 weeks. Surveys were administered electronically via text message (SMS) links or email. The study protocol is registered at clinicaltrials.gov (study number RADX-P-2407).

Recruitment and Retention: Participants were recruited online using a variety of digital channels, including social media platforms such as Facebook and Instagram, recruitment partners, consumer groups, and other types of online networks. To qualify for the study, all participants expressed a desire to improve their digestive health, which was a core inclusion criterion. This study was conducted entirely virtually, with no in-person visit, to simulate real-world conditions. Retention proved challenging due to the self-directed nature of the study and lack of in-person oversight. Participants were free to withdraw from the study at any time, and a portion did not complete the study. The attrition was accounted for in the data analysis. The investigator had the authority to terminate a participant for medical or compliance reasons, but no one was terminated for medical reasons in this study.

Inclusion and exclusion criteria: Inclusion criteria included adults (>21 years), with an expressed desire to improve their digestive health. Exclusion criteria included pregnancy, heavy drinking (>3 drinks per day), diagnosed kidney, liver, or cardiovascular disease, chemotherapy or immunotherapy, and specific medications including antibiotics. The inclusion and exclusion criteria were designed to ensure a study population representative of individuals with an interest in improving gut health while minimizing confounding variables that could interfere with study outcomes.

Materials: The active and placebo products were manufactured and provided to Radicle Science by NutriScience Innovations. The active product contained ButyraGen®, a patent-pending complex of tributyrin, Sunfiber® (partially hydrolyzed guar fiber), acacia fiber, and other inactive constituents. Participants in the active group received a daily dose of 200 mg ButyraGen®, containing 50% tributyrin (providing 100 mg of tributyrin per day) and approximately 45% fiber (providing 90 mg of prebiotic fiber per day). The placebo product primarily consisted of maltodextrin. Both the active and placebo powders were encapsulated in hydroxy propyl methyl cellulose (HPMC) capsules with identical inactive colorants used to ensure blinding. Each participant received a single bottle containing 45 capsules of their randomly assigned product (active or placebo), sufficient for the 6-week study. The active and placebo products were tested for potency, microbial contamination, heavy metals, pesticides and residual solvents to ensure safety and quality before distribution.

Questionnaires: Participants were enrolled based on an inclusion/exclusion criteria questionnaire. Following enrollment, all participants provided demographic information and then completed a diet questionnaire along with a series of health assessments, including alcohol and cannabinoid use. These health questionnaires were administered at baseline, weekly, and at the end of the study. The questionnaires used in this portion of the study included:

PROMIS™ Anxiety 4A questionnaire (Anxiety is self-reported in this questionnaire). The PROMIS™ Gastrointestinal Anxiety Scale is a validated tool used to assess anxiety in adults. It is part of the Patient-Reported Outcomes Measurement Information System (PROMIS™), which was developed by the National Institutes of Health (NIH).

PROMIS™ Depression 4A questionnaire (Depression is self-reported and not clinically validated).

Digestion-associated QOL Questionnaire (DQLQ) (This questionnaire includes both digestive and anxiety questions which are helpful in associating gut with brain).

Outcomes measurements: The primary outcome measure of the overall study was an assessment of gastrointestinal (GI) health, based on the responses to a collective of validated GI questionnaires. This involves different questions, results and analyses, and have been reported elsewhere. REF Anxiety and depression as measured by the PROMIS™ questionnaires and the DQLQ questionnaire were secondary outcomes of the study and are reported here.

Statistical Analysis: Statistical analysis was performed on the dataset using the Intent-To-Treat (ITT) protocol. Participants included in the analysis completed the baseline and at least one of the 6-week questionnaires. This approach was chosen to reflect a real-world setting where participants made independent decisions about completing questionnaires without in-person visits.

Given the substantial dropout early in the study, it was decided not to use the ITT protocol coupled with the Last Observation Carry Forward (LOCF) analysis of the data. Although LOCF is widely used and yielded similar results in preliminary testing, it effectively doubled the sample size for each questionnaire. However, it was determined that the ITT-LOCF was inferior and less robust than the ITT analysis without carry forward, as it artificially inflated the dataset without necessarily improving data integrity. Despite the lower participant count in the ITT analysis, it was found sufficiently powered to detect meaningful differences and provide insights.

The Minimal Clinically Important Difference (MCID) analysis was used to analyze the results. MCID is a participant-centered concept that considers the magnitude of improvement and how much value the patient places on the change. The MCID examines the likelihood of meaningful improvements in participant questionnaires by the end of the study. An MCID was defined as an improvement of at least 0.5 standard deviations from their baseline in the target health measure. A Poisson general linear model was used to fit survey data to predict the occurrence of an MCID, while controlling for covariates such as sex, age and BMI. The associated MCID risk ratio is computed to compare the likelihood of an improvement for the active versus placebo study arms. A risk ratio greater than 1.0 indicates a higher probability of achieving clinically significant improvement in the active group versus the placebo with a 95% confidence interval (C.I.). The MCID analysis also allows for statistical analysis of subsets of the populations, allowing for a deeper understanding of how specific population subgroups responded to the intervention. Since there was no significant difference between the active and placebo arms and the individual questions within the questionnaires are considered validated, further post-hoc statistical analysis was performed on individual questions, as well as the Digestion-associated Quality of Life (DQLQ) questions.

The more rigorous statistical approach best reflects the clinical outcomes. This statistical framework provides meaningful insights into the effects of ButyraGen® supplementation on gut-brain axis outcomes while accounting for real-world variability and consumer adherence.

A small number of participants reported cannabinoid use. The study found that there was no effect of cannabinoid use on the statistical analysis of the results presented here. The study allowed for real-world consumption of alcohol and caffeine without bounds.

### Results

Study Demographics: A total of 596 participants were enrolled in the study, with 229 completing the active arm and 218 completing the placebo arm. The study population consisted of 32% males and 68% females, with a mean age of 45 years (range: 21-76). Approximately 26% of the participants identified as racial minorities, and 37.4% self-reported having gastrointestinal (GI) disease. Participants were geographically diverse, representing 47 of 50 US states.

Study Compliance: Seventy-five percent of the enrolled participants completed the trial, reflecting high adherence in this real-world study format. Weekly survey completion rates ranged from 60% to 90%, with similar rates observed across both arms. The analysis of dropout shows a rapid dropout after the first week, which really sets the participant levels for the remainder of the study where the participant participation declines are less and provides more useful data.

Anxiety (PROMIS™ Anxiety 4A): Statistically significant improvements were observed in anxiety in men as measured by the PROMIS™ Anxiety 4A questionnaire and they are presented here. Male participants in the active group demonstrated a significantly higher likelihood (27.4%) of achieving an improvement compared to the placebo group (p=0.0074, risk ratio=2.14 (+/−0.28)). The smaller population of men who enrolled in the study led to a smaller population of men for analysis, but this still resulted in a statistically significant improvement in their anxiety. These results are shown in Table 1.

TABLE 1
Anxiety results for males

| Questionnaire | PROMIS ™ Anxiety 4A Men Subset |
|---|---|
| Total # male participants | 90 |
| Active male participants | 42 |
| Placebo male participants | 48 |
| MCID improvement p value | 0.0074 |
| MCID risk ratio (+/−95% C.I.) | 2.14 (+/−0.28) |
| Difference in overall MCID | 27.4% |
| (active − placebo) | |

There was no observation of statistically significant improvements in anxiety levels across the overall population (combined male and female participants). However, the results did show non-statistically significant improvements (trends) in the overall population of 9.3% (p=0.140 rr=1.24). There were no trends with females.

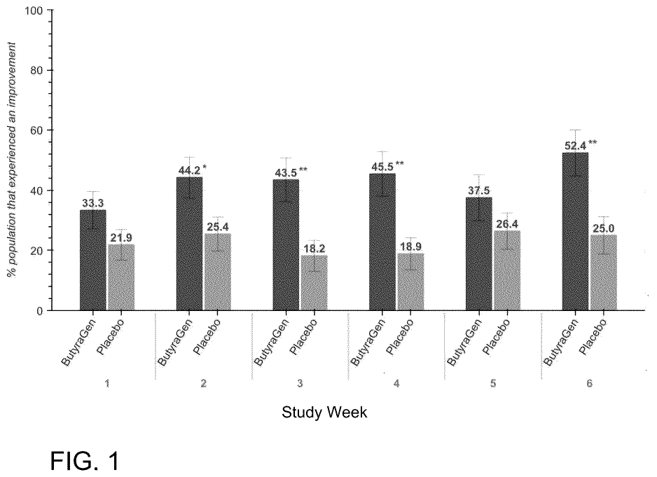

A weekly analysis of anxiety improvement, shown in FIG. 1, indicates that male participants taking ButyraGen® experienced a consistent greater reduction in anxiety compared to placebo throughout the study. Statistically significant differences were observed in weeks 3, 4, and 6 (p<0.01) and week 2 (p<0.05).

Anxiety improved rapidly (by week 2) and continued to improve with improved p values through the study (except for week 5).

In order to explore this anxiety relationship further through post-hoc analyses, we looked for statistically significant improvements at the question level. The individual questions are also validated by NIH as part of the validation of the overall questionnaire. The results are presented in Table 2.

TABLE 2
Analysis of the individual questions in the Anxiety 4A questionnaire

| Questionnaire | Anxiety 4A | Anxiety 4A | Anxiety 4A | Anxiety 4A |
|---|---|---|---|---|
| Question | “fearful” | “focus” | “overwhelmed” | “uneasy” |
| Population subset | None | Male | | All |
| Total # participants | 288 | 90 | | 288 |
| Active participants | 146 | 42 | | 146 |
| Placebo participants | 142 | 48 | | 142 |
| MCID improvement p | 0.0260 | 0.0048 | P > 0.05 | 0.0037 |
| value | | | | |
| MCID risk ratio | 1.44 (+/−0.16) | 2.20 (+/−0.29) | | 1.47 (+/−0.13) |
| (+/−95% C.I.) | | | | |
| Difference in overall | 12.9% | 29.5% | | 18.2% |
| MCID (active − placebo) | | | | |

The results for “focus” (I found it hard to focus on anything other than my anxiety) were similar to the overall group. However, two of the other questions “fearful” (I felt fearful) and “uneasy” (I felt uneasy) showed statistically significant responses for the entire population (both males and females). The “overwhelmed” question was not statistically significant for any meaningful subset population.

Depression (PROMIS™ Depression 4A): Similar to the Anxiety results, the Depression questionnaire did not show statistically significance for the total population. However, there was statistical significance observed for the sub-population of those who reported no GI disease (both men and women). The results are shown in Table 3.

TABLE 3
Depression results for those with no GI Disease

| | PROMIS ™ Depression 4A |
|---|---|
| Questionnaire | No GI Disease Subset |
| Total # participants | 214 |
| Active participants | 112 |
| Placebo participants | 102 |
| MCID improvement p value | 0.0110 |
| MCID risk ratio (+/−95% C.I.) | 2.00 (+/−0.27) |
| Difference in overall MCID | 15.6% |
| (active - placebo) | |

These results are notable, as “no GI disease” refers to participants who self-reported no GI-related diseases in their baseline survey with no other constraints applied. This broader inclusion allowed for a substantial subset population improvement. For the overall Depression 4A questionnaire, with no subpopulation constraints, there was non-statistically significant improvements trend of 7.0% (p=0.222 rr=1.29).

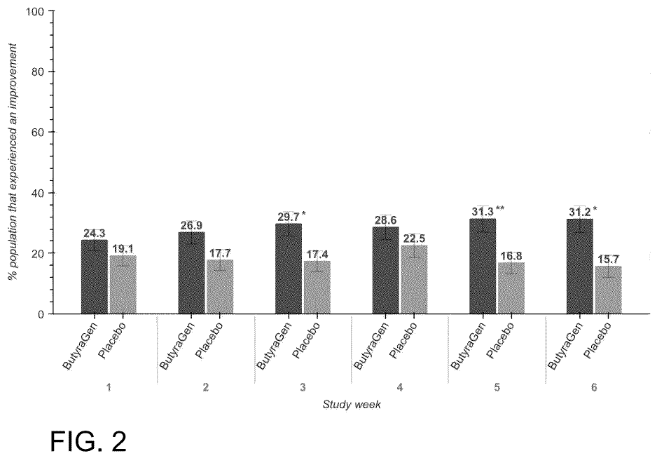

The analogous temporal results for self-reported depression are shown in FIG. 2. In this case the temporal effects were still significant by week 3 and at the end of the study in week 6. However, these depression temporal results were generally weaker in statistical correlation compared to the anxiety results.

In order to explore this depression relationship further through post-hoc analyses, we looked for statistically significant improvements at the individual question level. The individual questions are validated by NIH as part of the validation of the overall questionnaire. The results are presented in Tables 4, 5 and 6.

TABLE 4
Analysis of the individual questions in the Depression
4A questionnaire for those with No GI Disease

| | Depres- | Depres- | Depres- | Depres- |
|---|---|---|---|---|
| | sion | sion | sion | sion |
| Questionnaire | 4A | 4A | 4A | 4A |
| Question | “worthless” | “helpless” | “depressed” | “hopeless” |
| Population subset | No GI | No GI | No GI | No GI |
| | Disease | Disease | Disease | Disease |
| Total # participants | | 214 | 214 | 214 |
| Active participants | | 112 | 112 | 112 |
| Placebo participants | | 102 | 102 | 102 |
| MCID improvement p | p > 0.05 | 0.0124 | 0.0664 | 0.0074 |
| value | | | | |
| MCID risk ratio | | 1.68 (+/−0.21) | 1.73 (+/−0.20) | 1.93 (+/−0.25) |
| (+/−95% C.I.) | | | | |
| Difference in overall | | 16.6% | 18.3% | 18.0% |
| MCID (active − | | | | |
| placebo) | | | | |

TABLE 5
Analysis of the individual questions in the Depression
4A questionnaire for those with Age < 40 yrs.

| | Depres- | Depres- | Depres- | Depres- |
|---|---|---|---|---|
| | sion | sion | sion | sion |
| Questionnaire | 4A | 4A | 4A | 4A |
| Question | “worthless” | “helpless” | “depressed” | “hopeless” |
| Population subset | Age < 40 yrs | Age < 40 yrs | Age < 40 yrs | Age < 40 yrs |
| Total # participants | | 98 | 98 | 98 |
| Active participants | | 53 | 53 | 53 |
| Placebo participants | | 45 | 45 | 45 |
| MCID improvement p | p > 0.05 | 0.0223 | 0.400 | 0.0314 |
| value | | | | |
| MCID risk ratio | | 1.97 (+/−0.36) | 1.77 (+/−0.28) | 1.92 (+/−0.30) |
| (+/−95% C.I.) | | | | |
| Difference in overall | | 24.7% | 11.8% | 18.0% |
| MCID (active − | | | | |
| placebo) | | | | |

TABLE 6
Analysis of the individual questions in
the Depression 4A questionnaire for Males

| | Depres- | Depres- | Depres- | Depres- |
|---|---|---|---|---|
| | sion | sion | sion | sion |
| Questionnaire | 4A | 4A | 4A | 4A |
| Question | “worth- | “help- | “de- | “hope- |
| | less” | less” | pressed” | less” |
| Population subset | Male | Male | Male | Male |
| Total # participants | | | | 90 |
| Active participants | | | | 42 |
| Placebo participants | | | | 48 |
| MCID improvement | p > 0.05 | p > 0.05 | p > 0.05 | 0.0144 |
| p value | | | | |
| MCID risk ratio | | | | 2.67 (+/−0.40) |
| (+/−95% C.I.) | | | | |
| Difference in overall | | | | 23.5% |
| MCID (active − | | | | |
| placebo) | | | | |

The results from the post-hoc analysis of the individual questions in the Depression questionnaire showed statistical significance for some of the questions in participants with no GI disease, younger participants (<40 yrs) and again for males. No other significance with a subset total population>50 were found. As for the Anxiety questionnaire, statistical significance for those with No GI Disease is insightful and is discussed later.

DQLQ Questions: Two questions in the DQLQ questionnaire relating to psychological actions—“distracted” (I was distracted while doing certain activities”, and “social” (social activities-spending time with friends or family, going out to eat etc., were unpleasant or avoided) were the only two questions in the post-hoc analysis of the DQLQ questionnaire individual questions to show statistically significant improvement with a subset population>50. The results are listed in Table 7.

TABLE 7
Participants showing significant improvement
in their DQLQ questions

| DQLQ Questionnaire post-hoc analysis | “Distracted” | “Social” |
|---|---|---|
| Specific population | Baseline | All |
| | (sometimes/ | |
| | occasionally) | |
| Total # participants | 109 | 288 |
| Active participants | 53 | 146 |
| Placebo participants | 56 | 142 |
| MCID improvement p value | 0.0291 | 0.0340 |
| MCID risk ratio (+/−95% C.I.) | 1.25 (+/−0.10) | 1.39 (+/−0.16) |
| Difference in overall MCID | 17.2% | 12.2% |
| (active − placebo) | | |

Participants were significantly less distracted than at baseline for those who were distracted sometimes and occasionally at baseline. This question relates to physiological function exclusively. Participants (all subsets) also significantly improved their ability to socialize per the question. This question seems to relate to both GI function and physiological function.

Other results: Other post-hoc anxiety and depression statistical analyses were either not significant, marginally significant or were significant for smaller subset populations and are not considered here.

This study provides important new insights into the potential benefits of ButyraGen® supplementation on the gut-brain axis, highlighting its efficacy in specific populations and physiological measures. The results demonstrate that ButyraGen® supplementation at a clinically useful dose led to meaningful improvements in anxiety and depression, particularly in men, those without GI disease, and younger participants (<40 yrs). This suggests potential targeted applications for supporting brain function, managing psychological stress, and improving overall quality of life through the gut-brain axis. The response appears to be fast-acting, with statistically significant improvements observed in certain situations as early as 2 weeks, with more pronounced benefits by 6-weeks.

Before delving into the specific results, it is important to consider two potential pathways through which ButyraGen® may influence the gut-brain axis. The first pathway follows a cause-and-effect relationship, where physiological improvements in gut health-such as reduced digestive distress-lead to a corresponding reduction in anxiety and psychological discomfort. This mechanism likely involves gut-brain communication via the vagus nerve, biochemical signaling, or hormonal pathways, with butyrate produced by ButyraGen® playing a key role in mediating these effects. The second pathway suggests a more direct neurological impact, where butyrate generated from ButyraGen® influences brain function through established neurochemical pathways, independent of gut symptom relief. This route may contribute to reduced anxiety and improved mood through mechanisms already discussed. While the study did not include biochemical measurements due to the virtual trial format, participant responses allowed exploration of these potential gut-brain interactions. The findings suggest that ButyraGen® may engage both pathways, highlighting its dual role in gut and brain health and specifically on the gut-brain axis. These results may reflect the unique butyrate-generating abilities of ButyraGen® versus endogenous butyrate generation in the gut from normal fiber fermentation.

Anxiety Discussion: Anxiety (or anxiousness) is a common mental health condition linked to chronic stress, disrupted sleep, impaired cognitive function, and reduced executive processing. In this study, self-reported anxiety significantly improves in male participants following ButyraGen® supplementation, with a steady reduction in anxiety symptoms from week 2 onward and statistically significant improvements at weeks 3, 4, and 6. The rapid onset of action suggests that ButyraGen® may have both acute and sustained effects on psychological well-being. Anxiety in men can be caused by several factors, including work stress, family dynamics, traumatic events, and more. It also can stem from ongoing medical conditions like metabolic dysfunction and heart disease. Hormonal imbalances, particularly low testosterone levels, are also associated with increased anxiety and elevated cortisol levels, a key stress hormone. Interestingly, butyrate may be associated with higher testosterone in men, which could partially explain the observed improvements in anxiety. Additionally, post-hoc analysis of individual items from the Anxiety 4A questionnaire revealed all participants (men and women) showed statistically significant improvements in being fearful and uneasy, while male participants specifically showed statistically significant improvements in focus. This suggests that ButyraGen® may support psychological balance in both males and females (all participants) but its impact on anxiety appears to be more pronounced in men.

Depression Discussion: It is important to note that the Depression 4A questionnaire in this study relied on self-reported assessment and was not a formal clinical diagnosis. The factors showing improvements in depression were related to being fearful, unfocused, or uneasy. Depression appeared to correlate most strongly with participants who did not have self-reported GI disease, with statistically significant improvements observed in this subgroup. This suggests that, for these participants, the improvements in depression were not directly related to improvements in GI disease. This may be related to the second pathway discussed above—where ButyraGen® generates butyrate independent of GI disease and impacts psychological and brain health.

In the post-hoc analysis, the no GI disease subgroup continued to show improvements in depression related symptoms. Additionally, younger participants (<40) reported statistically significant improvements in feelings of helplessness, hopelessness, and depression. This could be attributed to better metabolic health in younger individuals, allowing butyrate to be more effectively utilized for neurological and psychological benefits rather than digestive health. Due to the limited sample size, we did not perform further analysis on multi-factorial subgroups, as the reduced participant count would impact statistical robustness.

DQLQ Discussion: As mentioned in the results section, the two questions in the DQLQ questionnaire (“distracted” and “social”) may offer insights into the two gut-brain axis pathways. The social parameter may reflect the first pathway, where butyrate generation improves gut health, leading to better psychological outcomes. The distracted parameter may correspond to the second pathway, where butyrate generation directly influences cognitive function. We have not seen this aspect of gut-brain axis health discussed previously, likely because previous studies focus on either gut microbiota driven butyrate production or direct exogenous butyrate supplementation. Our study is unique in that ButyraGen® serves as a butyrate generator which may improve both gut and brain health.

This study was conducted entirely virtually with no in-clinic visits, representing real-world conditions and a diverse U.S. population. The use of validated tools such as PROMIS™ and DQLQ helped ensure a robust and reliable set of questions. The large starting participant population was critical to obtaining sufficient ending populations in order to demonstrate statistical significance as a result of dropout, especially after week-1. The findings demonstrate statistically significant improvements in physiological health over the 6-week course and 200 mg ButyraGen® dose regimen. The study supported the safety and tolerability of ButyraGen® in a general population.

The present disclosure and methodology demonstrates an ability of ButyraGen®, a direct butyrate generating supplement, to influence the gut-brain axis, through two pathways-butyrate generation leading to improvements in gut health which lead to improvements in brain health, and direct butyrate generation in the gut directly leading to improvements in brain health, with a fast-acting response. By addressing common digestive and brain health concerns, ButyraGen® represents a promising intervention for promoting gut-brain axis health and enhancing overall well-being in diverse populations.

It should be noted that various changes and modifications to the presently preferred embodiments described herein will be apparent to those skilled in the art. Such changes and modifications may be made without departing from the spirit and scope of the present invention and without diminishing its attendant advantages. Further, references throughout the specification to “the invention” are nonlimiting, and it should be noted that claim limitations presented herein are not meant to describe the invention as a whole. Moreover, the invention illustratively disclosed herein suitably may be practiced in the absence of any element which is not specifically disclosed herein.

1. A method for supplementing butyrate in a diet of a mammal to promote improved physiological outcomes, the method comprising the steps of:
providing a dietary supplement composition formulated to increase generation of butyrate in a gut of the mammal;
administering the dietary supplement composition to the mammal, wherein the dietary supplement composition produces a therapeutically effective increase in the amount of butyrate in the gut of the mammal for improved physiological outcomes.

2. The method for supplementing butyrate of claim 1, wherein administering the dietary supplement composition to the mammal includes providing the dietary supplement in a daily dosage of 200 mg.

3. The method for supplementing butyrate of claim 2, wherein the dietary supplement composition comprises:
between about 30 and 70% tributyrin;
between about 5 and 15% partially hydrolyzed guar gum fiber; and
between about 15-65% other ingredients.

4. The method for supplementing butyrate of claim 2, wherein the dietary supplement composition comprises:
tributyrin provided in a dosage amount of between 10 and 100 mg per day; and,
partially hydrolyzed guar gum fiber provided in a dosage amount of between 0.7 and 50 mg per day.

5. The method for supplementing butyrate of claim 1, wherein administering the dietary supplement composition to the mammal produces a therapeutically effective increase in the amount of butyrate in the gut of the mammal to reduce gastrointestinal disorders in the mammal.

6. The method for supplementing butyrate of claim 5, wherein the step of reducing gastrointestinal disorders in the mammal correlates with a reduction in anxiety and psychological discomfort in the mammal.

7. The method for supplementing butyrate of claim 5, wherein the dietary supplement composition modulates a gut-brain axis in the mammal.

8. The method for supplementing butyrate of claim 1, wherein administering the dietary supplement composition to the mammal produces a therapeutically effective increase in the amount of butyrate in the gut of the mammal to influence brain function of the mammal.

9. The method for supplementing butyrate of claim 8, wherein administering the dietary supplement composition to the mammal produces a therapeutically effective increase in the amount of butyrate in the gut of the mammal to influence brain function of the mammal independent from gastrointestinal disorders in the mammal.

10. The method for supplementing butyrate of claim 9, wherein the dietary supplement composition modulates a gut-brain axis in the mammal independent of gastrointestinal disorders relief to improve psychological outcomes.
